(19) United States
(12) Patent Application Publication
(10) Pub. No.: US 2004/0171090 A1
(43) Pub. Date: Sep. 2, 2004

(54) Anti-tumor antigen against HTLV-I tumor or antigen epitope thereof

(76) Inventors: Shino Hanabuchi, Houston, TX (US); Takashi Ohashi, Tokyo (JP); Mari Kannagi, Tokyo (JP)

Correspondence Address: FROMMER LAWRENCE & HAUG, 745 FIFTH AVENUE- 10TH FL., NEW YORK, NY, 10151, US

(21) Appl. No.: 10/704,229

(22) Filed: Nov. 7, 2003

Related U.S. Application Data
(63) Continuation-in-part of application No. PCT/JP02/04406, filed on May 2, 2002.

(30) Foreign Application Priority Data
May 8, 2001 (JP) 2001-137526

(51) Int. Cl. G01N 33/574; C07K 14/705

(52) U.S. Cl. 435/7.23; 530/350

(57) ABSTRACT

The present invention provides a immunogenic composition for inducing an immune response containing a CTL recognition antigen or an antigen epitope thereof, which can be obtained by screening a CTL recognition antigen or an antigen epitope thereof having an anti-tumor effect against HTLV-I tumors such as ATL, or a DNA that encodes them as an active ingredient and the like. Dominant epitope GAFLTNVPY was identified by the following steps: splenic T cells derived from immunocompetent rats immunized with HTLV-I-infected cell lines were stimulated with formalin-fixed HTLV-I-infected cell lines; HTLV-I-specific CTL cell lines were established; cytotoxic activities of the above-mentioned HTLV-I-specific CTL cell lines against target cells G14 sensitized with synthetic peptides which are candidate for an epitope were measured. By immunizing immunocompetent rats with said epitope synthetic peptide and an adjuvant, then inducing tumor antigen epitope-specific CTLs, the proliferation of HTLV-I-infected tumor cells in vivo can be suppressed.

### REFERENCE TO RELATED APPLICATIONS

This application is a continuation-in-part of International Application PCT/JP02/04406 filed on May 2, 2002 and published as WO 02/090981 A1 on Nov. 14, 2002, which application claims priority from Japanese Application No. 2001-137526 filed May 8, 2001.

Each of the foregoing applications, and each document cited or referenced in each of the foregoing applications, including during the prosecution of each of the foregoing applications and (“application cited documents”), and any manufacturer 's instructions or catalogues for any products cited or mentioned in each of the foregoing applications and articles and in any of the application cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

It is noted that in this disclosure and particularly in the claims, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

### FIELD OF THE INVENTION

The present invention relates to a screening method for a CTL recognition antigen or an antigen epitope thereof which can induce cytotoxic T lymphocytes (CTLs) having an anti-tumor effect against human T cell leukemia virus type I (HTLV-I) tumors such as adult T cell leukemia (ATL), etc.; a screening method for an adjuvant that enhances the inducing activity of CTLs having an anti-tumor effect against HTLV-I tumors by a CTL recognition antigen or an antigen epitope thereof or an antigen epitope peptide thereof which can induce CTLs having an anti-tumor effect against HTLV-I tumors; an immunogenic composition and the like for inducing an immune response containing said CTL recognition antigen or antigen epitope thereof, or DNA thereof as an active ingredient.

### BACKGROUND OF THE INVENTION

HTLV-I is involved in the pathogenesis of ATL, HTLV-I-associated myelopathy/tropical spastic paraparesis (HAM/TSP), as well as other inflammatory diseases (Blood 50, 481492, 1977; Proc. Natl. Acad. Sci. USA 77, 7415-7419, 1980; Proc. Natl. Acad. Sci. USA 78, 6476-6480, 1981; Lancet. 2, 407410, 1985; Lancet. 1, 1031-1032, 1986). HTLV-I is considered to have a specific sequence called pX between its env and 3′LTR, wherein this pX region cannot be seen in carcinogenic retroviruses of other animals, and its production called Tax protein is considered to play an important role for various pathogenesis of HTLV-I. It is also known that said HTLV-I Tax is a virus control protein and can immortalize rat and human cells in vitro (Proc. Natl. Acad. Sci. USA 87, 1071-1075, 1990; Blood 86, 4243-4249, 1995; J. Virol. 66, 4570-4575, 1992). These findings strongly indicate that HTLV-I Tax is involved in the mechanisms of HTLV-I-induced leukemogenesis. However, the mechanisms of pathogenesis of ATL in vivo are still unclear.

A number of studies have revealed that the levels of host cellular immunity against HTLV-I in ATL patients differ from those in HAM/TSP patients, and that HTLV-I-specific CTLs can be found in HAM/TSP patients and asymptomatic HTLV-I carriers but CTLS are only rarely found in ATL patients (J. Immunol. 130, 2942-2946, 1983; J. Exp. Med. 158, 994-999, 1983; J. Immunol. 133, 1037-1041, 1984; Nature 348, 245-248, 1990; Virology 188, 628-636, 1992; Int. J. Cancer 54, 582-588, 1993). Based on these observations, it is considered that host cellular immunity may influence pathogenesis of HTLV-I-associated diseases.

In general, the above-mentioned CTLs play an important role not only in viral clearance, but also in tumor eradication. It is reported by the present inventors and other groups that HTLV-I-specific CD8⁺ CTLs in HTLV-I carriers recognize HTLV-I Tax (Nature 348, 245-248, 1990; Int. Immunol. 3, 761-767, 1991), and lyse ATL cells in vitro (J. Immunol. 133, 1037-1041, 1984; Int. J. Cancer 54, 582-588, 1993). These observations indicate that HTLV-I-specific CTLs may be an important effector of host immunosurveillance against HTLV-I-induced tumor proliferation. However, it is still unclear whether the above-mentioned role of CTLs is against HTLV-I tumors in vivo or merely a consequence of infection.

To clarify the effect of CTLs on HTLV-I leukemogenesis in vivo, the present inventors developed two experimental rat model systems of ATL-like diseases (J. Virol. 73, 6031-6040, 1999; J. Virol. 74, 428-435, 2000; Japanese Patent Application No.H10-315174). One is a model of T cell lymphomas in athymic rats following inoculation of HTLV-I-immortalized rat cell lines (J. Virol. 73, 6031-6040, 1999). In this model, adoptive transfer of immune T cells protected rats from fatal lymphomas. In the other model, the development of T cell lymphomas was induced by treatment with anti-CD80 and -CD86 monoclonal antibodies which block costimulatory signals for T cell activation in immunocompetent rats (J. Virol. 74, 428-435, 2000). These findings strongly indicate that the role of a host T cell immune response as to prevent the proliferation of HTLV-I tumors in vivo.

The observations made in studies of CTLs in human HTLV-I-infected patients and the above-mentioned rat models indicate that augmentation of HTLV-I-specific CTLs in pre-ATL patients might protect them from pathogenesis of ATL. Since the recovery rate of ATL is extremely low among lymphoproliferative disorders because of its resistance to chemotherapy, the development of therapies by immunological approach at an early stage of diseases have been expected. In order to induce an effective anti-tumor immune response, the tumor antigen must be precisely clarified and a strong immunogen recognized by cellular immunity must be administered to the host. In particular, to induce a CD8⁺ CTL response which is one of the major populations that recognize tumors, presentation of an appropriate peptide processed by MHC class I as well as class II antigens of antigen-presenting cells is essential (Nature 343, 692-696; 1989). Because of the preference of the pathway of antigen presentation by MHC class I, a variety of viral vectors have been proposed for delivery and expression of exogenous genes that encode target antigens (J. Natl. Cancer Inst. 90, 1894-1900, 1998). It is considered that similar effects can be obtained by the direct injection of untreated DNA (Annu. Rev. Immunol. 18, 927-974, 2000). In addition, peptide-based vaccines corresponding to CTL epitopes that directly bind to MHC molecules, which are safer than DNA-based vaccines, are also under consideration (Int. J. Cancer 63, 883-885, 1995).

ATL is a neoplastic disease caused by the infection of HTLV-I, which shows high carrier rate in Japan, however, it has been considered to be a malignant tumor with extremely bad prognosis because of its resistance to chemotherapeutic agents. On the other hand, various clinical observations indicate the anti-tumor effect of host cellular immunity, particularly of CTLs. The object of the present invention is to provide: a screening method for a CTL recognition antigen or an antigen epitope thereof which can induce CTLs having an anti-tumor effect against HTLV-I tumors such as ATL, etc.; a screening method for an adjuvant that enhances the inducing activity of CTLs having an anti-tumor effect against HTLV-I tumors by a CTL recognition antigen or an antigen epitope thereof or an antigen epitope peptide thereof which can induce CTLs having an anti-tumor effect against HTLV-I tumors; a immunogenic composition and the like for inducing an immune response containing said CTL recognition antigen or antigen epitope thereof, or DNA thereof as an active ingredient.

### SUMMARY OF THE INVENTION

The present inventors made a keen study to attain the object mentioned above. Splenic T cells derived from syngeneic immunocompetent rat immunized with nu/+ rat-derived HTLV-I-infected cell lines, FPM1-V1AX, were repeatedly stimulated every 2 weeks by using formalin-fixed FPM1-V1AX to establish HTLV-I-specific CTL cell lines, and the cytotoxic activity of the above-mentioned HTLV-I-specific CTL cell lines against target cells G14 sensitized with various synthetic peptides that are considered to include the dominant recognition epitope of HTLV-I-specific CTLs were studied, and the present inventors identified a peptide for which target cells show particularly strong CTL sensitivity, namely the dominant epitope of a CTL recognition antigen which can induce CTLs having an anti-tumor effect against HTLV-I tumors. The present inventors verified that it is possible to induce tumor antigen epitope-specific CTLs in immunocompetent rats by using synthetic peptides of said epitope as an immunogen and using an adjuvant, and that said induced CTLs can strongly suppress proliferation of HTLV-I-infected tumor cells in vivo. Thus, the present invention has completed.

The present invention relates to: a screening method for a CTL recognition antigen or an antigen epitope thereof, which can induce cytotoxic T lymphocytes (CTLs) having an anti-tumor effect against HTLV-I tumors, wherein CTLs induced by a test substance is administered to a non-human animal model of HTLV-I-associated disease, and the change of tumors in said non-human animal is measured and assessed; the screening method for a CTL recognition antigen or an antigen epitope thereof, which can induce CTLs having an anti-tumor effect against HTLV-I tumors according to claim 1 or paragraph 1, wherein the non-human animal model of HTLV-I-associated disease is a non-human animal model of adult T cell leukemia; the screening method for a CTL recognition antigen or an antigen epitope thereof which can induce CTLs having an anti-tumor effect against HTLV-I tumors according to claim 1 or paragraph 1, wherein the non-human animal is a rat; a screening method for a CTL recognition antigen or an antigen epitope thereof which can induce CTLs having an anti-tumor effect against HTLV-I tumors, wherein CTLs induced by a test substance is contacted with HTLV-I-infected tumor cell lines, and the cytotoxic activity of said CTLs is measured and assessed; a screening method for a CTL recognition antigen or an antigen epitope thereof which can induce CTLs having an anti-tumor effect against HTLV-I tumors, wherein target cells sensitized with a test substance or target cells that express a test substance are contacted with HTLV-I-specific CTL cell lines, and the cytotoxic activity of said HTLV-I-specific CTL cell lines is measured and assessed; the CTL recognition antigen or the antigen epitope thereof which can induce CTLs having an anti-tumor effect against HTLV-I tumors, which is obtained by the screening method of claim 1 or paragraph 1; the antigen epitope according to claim 4 or paragraph 6, wherein the antigen epitope is a peptide comprising an amino acid sequence shown by Seq. ID No.2.

The present invention further relates to: a screening method for an adjuvant that enhances an anti-tumor effect against HTLV-I tumors, wherein CTLs induced by using the CTL recognition antigen or the antigen epitope thereof according to claim 4 or 5; or paragraph 6 or 7, and a test adjuvant are administered to a non-human animal model of HTLV-I-associated disease and the change of tumors in said non-human animals is measured and assessed; the screening method for an adjuvant that enhances an anti-tumor effect against HTLV-I tumors according to claim 6 or paragraph 8, wherein the non-human animal model of HTLV-I-associated disease is a non-human animal model of adult T cell leukemia; the screening method for an adjuvant that enhances an anti-tumor effect against HTLV-I tumor according to claim 6 or paragraph 8, wherein the non-human animal is a rat; a screening method for an adjuvant that enhances an anti-tumor effect against HTLV-I tumors, wherein CTLs induced by using the CTL recognition antigen or the antigen epitope thereof according to claims 4 or 5; or paragraph 6 or 7; and a test adjuvant are contacted with HTLV-I-infected tumor cell lines, and the cytotoxic activity of said CTLs is measured and assessed.

The present invention still further relates to: an immunogenic composition for inducing an immune response containing a CTL recognition antigen or an antigen epitope thereof which can induce CTLs having an anti-tumor effect against HTLV-I tumors and which can be obtained by the screening method according to claim 1 or paragraphs 1-5; an immunogenic composition for inducing an immune response containing a DNA that encodes a CTL recognition antigen or an antigen epitope thereof which can induce CTLs having an anti-tumor effect against HTLV-I tumors obtained by the screening method according to claim 1 or paragraphs 1-5; the immunogenic composition for inducing an immune response according to claim 9 or 10; or paragraph 13 or 14, wherein the CTL recognition antigen is an HTLV-I Tax protein shown by Seq. ID No.1; the immunogenic composition for inducing an immune response according to claim 11 or paragraph 15, wherein the CTL recognition antigen is a protein comprising an amino acid sequence wherein at least one amino acid is deleted, substituted, or added in the amino acid sequence shown by Seq. ID No.1, which can induce CTLs having an anti-tumor action against HTLV-I tumors; the immunogenic composition for inducing an immune response according to claim 9 or 10; or paragraph 13 or 14, wherein the CTL recognition antigen epitope is a peptide comprising the amino acid sequence shown by Seq. ID No.2; the immunogenic composition for inducing an immune response according to claim 13 or paragraph 17, wherein the CTL recognition antigen epitope is a peptide comprising an amino acid sequence wherein at least one amino acid is deleted, substituted, or added in the amino acid sequence shown by Seq. ID No.2, which can induce the CTLs having an anti-tumor action against HTLV-I tumors; the immunogenic composition for inducing an immune response according to claims 9 or 10; or paragraph 13 or 14, further containing an adjuvant that enhances an anti-tumor effect against HTLV-I tumors; the immunogenic compositon for inducing an immune response according to claim 15 or paragraph 19, wherein the adjuvant is obtained by the screening method according to claim 6 or paragraphs 8-12; the immunogenic composition for inducing an immune response according to claim 15 or paragraph 19, wherein the adjuvant is ISS-ODN; an HTLV-I recognition CTL induced by the immunogenic composition for inducing an immune response according to any of claims 9 to 17 or paragraphs 13 to 21; and a pharmaceutical composition containing the HTLV-I recognition CTL according to claim 18 or paragraph 22.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 is a figure showing the results of examining a subset of immune T cells considered to be directly required for the regression of tumors in vivo.

FIG. 2 is a figure showing the results of cytotoxic activities against target cells of various splenic T cells derived from rats wherein tumors have been completely regressed.

FIG. 3 is a figure showing the results of examining viral antigens recognized by HTLV-I-specific CTLs.

FIG. 4 is a figure showing the results of inhibition of cytotoxicity against target cells of HTLV-I-specific CTL cell lines in the presence of a competitor.

FIG. 5 is a figure showing the results of cytotoxic activities of various CTLs induced by HTLV-I-infected tumor cell line FPM1-V1AX.

FIG. 6 is a figure showing the results of screening of HTLV-I-specific CTL cell line recognition peptides.

FIG. 7 is a figure showing the results of screening of HTLV-I-specific CTL cell line recognition peptides in the case where long-tern-cultured CTL cell lines are used.

FIG. 8 is a figure showing the results of screening 9 amino acid synthetic peptides recognized by HTLV-I-specific CTL cell lines.

FIG. 9 is a figure showing the results of examining the cytotoxic activity of CTLs against target cells sensitized with various concentrations of peptides.

FIG. 10 is a figure showing the specificity of HTLV-I-specific CTL cell lines against synthetic peptide Tax 180-188.

FIG. 11 is a figure showing the results of proliferation suppression effect on HTLV-I tumors in vivo by Tax 180-188 recognition CTL cell lines.

FIG. 12 is a figure showing the effectiveness of Tax 180-188 and ISS-ODN as immunogenic compositions.

### DETAILED DESCRIPTION OF THE INVENTION

For ease of reference a summary of the accompanying sequence listings is given below:

SEQ ID NO:1 shows the amino acid sequence of an HTLV-I Tax protein.

SEQ ID NO:2 shows the amino acid sequence of an HTLV-I anti-tumor CTL recognition epitope.

SEQ ID NO:3 shows the amino acid sequence of another HTLV-I anti-tumor CTL recognition epitope.

SEQ ID NO:4 shows the amino acid sequence of an HTLV-I anti-tumor CTL recognition epitope.

SEQ ID NO: 5 shows a nucleic acid sequence of the adjuvant ISS-ODN.

SEQ ID NO: 6 shows an amino acid sequence of an influenza A viral epitope.

The screening method for a CTL recognition antigen or an antigen epitope thereof which can induce CTLs having an anti-tumor effect against HTLV-I tumors of the present invention can be particularly exemplified by the following: a method wherein CTLs induced by a test substance are administered to a non-human animal model of HTLV-I-associated disease such as ATL and the like, and the change of tumors in said non-human animal is measured and assessed; a method wherein CTLs induced by a test substance is contacted with HTLV-I-infected tumor cell lines, and cytotoxic activity of said CTLs is measured and assessed; a method wherein target cells sensitized with a test substance or target cells expressing a test substance is contacted with HTLV-I-specific CTL cell lines, and the cytotoxic activity of said HTLV-I-specific CTL cell lines is measured and assessed. As used herein, the CTL recognition antigen or the antigen epitope thereof, which can induce CTLs means a CTL recognition antigen or an antigen epitope thereof which can induce CTLs in vivo and in vitro.

The above-mentioned non-human animal model of HTLV-I-associated disease is not particularly restricted as long as it is a non-human animal which induces HTLV-I-associated diseases such as ATL, HAM/TSP, HAAP, uveitis, alveobronchiolitis, sialadenitis akin to Sjögren syndrome, etc., by infection of HTLV-I. However, the one that can reproducibly proliferate HTLV-I-infected tumor cells over long-term period is preferable. Besides, the non-human animal of the present invention can be particularly exemplified by non-human mammals such as mice, rats, guinea pigs, monkeys, cats, dogs, horses, cattle, or rabbits, but they are not limited to these examples. The method of generating a non-human animal model of ATL will be explained below with an ATL rat model as an example.

An ATL rat model can be obtained by, for instance, administering HTLV-I-infected tumor cell lines to an immunocompetent rat. However, an ATL rat model which can be obtained by administering HTLV-I-infected tumor cell lines subcutaneously, intraperitoneally, intravenously or the like to a non-human animal which is deficient in T cell function is more preferable in view of the reproducibility, and the point that HTLV-I-infected tumor cells can be proliferated and subcultured in vivo. As for a wild type rat used for evaluation, it is preferable to use a wild type rat syngeneic to an ATL rat model. The above-mentioned non-human animal, which is deficient in T cell function can be particularly exemplified by a nude non-human animal, including, for example, severe combined immunodeficiency mice (SCID) and National Institutes of Health nude rat F344/N Jcl-rnu/rnu, and the like, but they are not limited to these examples. In addition, there is no limitation to the above-mentioned HTLV-I-infected tumor cell lines as long as they are infected with HTLV-I by a known method and of which MHCs coincide with a wild type non-human animal. For instance, it can be particularly exemplified by cell lines such as FPM1-V1AX and the like.

The test substance used for the screening methods of the present invention can be particularly exemplified by proteins, peptides, DNAs, RNAs, antisense DNAs, antisense RNAs, etc. There is no limitation to the target cells sensitized with the above-mentioned test substance or the target cells that express a test substance as long as the MHCs thereof coincide, however, it can be preferably exemplified by G14 cells, which are CD8⁺ T cell lines (J. Virol. 74, 9610-9616, 2000).

There is no limitation to the expression system for preparation of cells that express a test substance as long as the expression system is capable of expressing the above-mentioned test substance intracellularly and the examples include, chromosome-, episome-, and virus-derived expression systems, for instance, vectors derived from bacterial plasmid, vectors derived from yeast plasmid, vectors derived from papovavirus such as SV40, vaccinia virus, adenovirus, fowlpox virus, pseudorabies virus, retrovirus, and vectors derived from bacteriophage or transposon and vectors derived from the combination of these two, e.g. vectors derived from genetic factors of plasmid and bacteriophage, such as cosmid and phagemid. This expression system may contain a regulatory sequence that not only induces expression but also regulate the expression. In addition, an expression vector series, which is capable of translating by changing the reading frame can also be used advantageously.

The expression system wherein said test substance is incorporated can be introduced into a host cell by the methods described in many standard laboratory manuals such as manuals of Davis et al. (BASIC METHODS IN MOLECULAR BIOLOGY, 1986) and of Maniatis et al. (MOLECULAR CLONING: A LABORATORY MANUAL, 2nd Ed., Cold Spring Harbor Laboratory Press, Cold Spring Harbor, N.Y., 1989), and can be conducted by the examples including calcium phosphate transfection, DEAE-dextran-mediated transfection, transvection, microinjection, cationic lipid-mediated transfection, electroporation, transduction, scrape loading, ballistic introduction, infection, etc.

There is no particular limitation to the HTLV-I-specific CTL cell lines of the present invention as long as they are the cell lines that specifically recognize HTLV-I, however, the cell lines restrained by MHC class I are more preferable. Said HTLV-I-specific CTL cell lines restrained by MHC class I can be obtained by a known method, for instance, a method wherein non-human animal-derived T cells that express MHC class I molecules are infected with HTLV-I, and proliferated in the body of a non-human animal which is deficient in T cell function (nude non-human animal), to establish HTLV-I-infected tumor cell lines, then the splenic T cells of the immunocompetent non-human animal which is immunized with such established cell lines are repeatedly stimulated with formalin-fixed HTLV-I-infected tumor cell lines.

The CTL recognition antigen which can be obtained by the above-mentioned screening method and which is capable of inducing CTLs having an anti-tumor effect against HTLV-I tumors can be exemplified by an HTLV-I Tax protein shown by Seq. ID No.1, a protein comprising an amino acid sequence wherein at least one amino acid is deleted, substituted, or added in the amino acid sequence shown by Seq. ID No.1 and which is capable of inducing CTLs having an anti-tumor action against HTLV-I tumors or the like. The antigen epitope which can be obtained by the above-mentioned screening method can be particularly exemplified by a peptide comprising an amino acid sequence shown by Seq. ID Nos.2, 3, or 4, or particularly preferably, a peptide comprising an amino acid sequence shown by Seq. ID No.2, a peptide comprising an amino acid sequence wherein at least one amino acid is deleted, substituted, or added in these amino acid sequences and which is capable of inducing CTLs having an anti-tumor action against HTLV-I tumors or the like, however, such antigen epitopes are not limited to these examples.

Therefore, the invention further comprehends polynucleotides that express such homologous polypeptides; and the corresponding degrees of homology or identity of those polynucleotides to polynucleotides encoding polypeptides to which homologous polypeptides have homology or identity of at least 50% to 100%. Homologous polypeptides advantageously contain one or more epitopes of the polypeptide to which there is identity or homology, such that homologous polypeptides exhibit immunological similarity or identity to the polypeptide to which there is identity or homology, e.g., the homologous polypeptide elicits similar or better immune response (to the skilled immunologist) than polypeptide to which there is identity or homology and/or the homologous polypeptide binds to antibodies elicited by and/or to which the polypeptide to which there is identity or homology binds, advantageously and not to other antibodies.

Accordingly, fragments of homologous polypeptides and of polypeptides to which there is identity or homology, advantageously those fragments which exhibit immunological similarity or identity to homologous polypeptides or polypeptides to which there is identity or homology, are envisoned as being expressed, and therefore, polynucleotides therefor which may represent fragments of polynucleotides of homologous polypeptides and of polypeptides to which there is identity or homology, are also envisioned by and useful in the instant invention.

The screening method for an adjuvant that enhances an anti-tumor effect against HTLV-I tumors of the present invention, namely, an adjuvant which is capable of inducing CTLs specific to a CTL recognition antigen or an antigen epitope thereof having a more efficient anti-tumor effect against HTLV-I tumors is not particularly restricted as long as such method includes: a method wherein CTLs induced by using the above-mentioned CTL recognition antigen or an antigen epitope thereof, or preferably, an antigen epitope peptide comprising the amino acid sequence shown by Seq. ID No.2 and a test adjuvant are administered to a non-human animal model of HTLV-I-associated disease such as adult T cell leukemia non-human animal model and the like, and the change of tumors in said non-human animal is measured and assessed; a method wherein CTLs induced by using the above-mentioned CTL recognition antigen or the antigen epitope thereof, or preferably, CTLs induced by using an antigen epitope peptide comprising the amino acid sequence shown by Seq. ID No.2 and a test adjuvant is contacted with HTLV-I-infected tumor cell lines, and cytotoxic activity of said CTLs is measured and assessed. The adjuvant which can be obtained by such screening method can be particularly exemplified by the following examples: ISS-ODN that includes CpG motif which is capable of efficiently inducing peptide-specific CTLs (Immunostimulatory DNA sequences-oligodeoxynucleotide; Nat. Med. 3, 849-854, 1997), QS21 that stimulates cytotoxic T cells (Quillaia saponaria; which can be commercially obtained from Cambridge Biotech, Worcester, Mass.), aluminum hydroxide, aluminum phosphate, aluminum oxide, oil-based emulsions, complete and incomplete Freund's adjuvant, saponin, vitamin E lysate, RIBI, ISCOM, among others.

The CTL recognition antigen or the antigen epitope thereof, or DNA encoding them which can be obtained by the aforementioned screening method and which can induce CTLs having an anti-tumor effect against HTLV-I tumors can be used as a immunogenic composition for inducing an immune response such as cellular immunity, humoral immunity and the like. The immunogenic composition for inducing an immune response of the present invention can be exemplified by a CTL recognition antigen which can induce CTLs having an anti-tumor effect against HTLV-I tumors, of which examples include: an HTLV-I Tax protein shown by Seq. ID No.1; a protein comprising an amino acid sequence wherein one or a few amino acids are deleted, substituted, or added in the amino acid sequence shown by Seq. ID No.1 and which can induce CTLs having an anti-tumor action against HTLV-I tumors, or a CTL recognition antigen epitope, of which examples include: an immunogenic composition for inducing an immune response containing a peptide comprising the amino acid sequence shown by Seq. ID No.2, or a peptide comprising an amino acid sequence wherein one or a few amino acids are deleted, substituted, or added in the amino acid sequence shown by Seq. ID No. 2, and which can induce CTLs having an anti-tumor action against HTLV-I tumors as an active ingredient, or an immunogenic composition for inducing an immune response containing DNA that encodes the above-mentioned CTL recognition antigen and the antigen epitope thereof which can induce CTLs having an anti-tumor action against HTLV-I tumors as an active ingredient. As for the immunogenic composition for inducing an immune response of the present invention, the one containing various adjuvants that further enhances cellular or local immunity is preferable. Said adjuvant can be exemplified by the one can be obtained by a screening method for an adjuvant that enhances an anti-tumor effect against the aforementioned HTLV-I tumors such as ISS-ODN and the like. When an adjuvant is used, it can also be used as a recombinant fusion protein or a recombinant fusion peptide generated from DNA which continuously encodes various fungus components, toxins and the like to be used as an adjuvant together with the above-mentioned CTL recognition antigen or an antigen epitope thereof, preferably an antigen epitope peptide comprising the amino acid sequence shown by Seq. ID No.2.

The immunogenic composition for inducing an immune response of the present invention may contain a pharmaceutically acceptable carrier or a diluent, an immunostimulator, an additive agent, etc. The carrier or diluent can be particularly exemplified by the following: a stabilizing agent such as SPGA; carbohydrates such as sorbitol, mannitol, starch, sucrose, glucose, dextran and the like; proteins such as albumin, casein and the like; protein-containing substances such as bovine sera, skim milk and the like; buffer solutions such as phosphate buffer solution, physiological saline, water and the like. The immunostimulator can be particularly exemplified by cytokines such as interleukin-2 (IL-2), interleukin-12 (IL-12), tumor necrosis factor α (TNF-α) and the like. The additive agent can be exemplified by polypeptides of low molecular weight (less than approximately 10 residues), proteins, amino acids, carbohydrate containing glucose or dextran, chelating agents such as EDTA and the like, protein stabilizing agents, inhibitors or suppressors of microorganism proliferation and the like, but they are not limited to these examples.

The pharmaceutical agents or pharmaceutical compositions provided by the present invention can be exemplified by the one containing HTLV-I recognition CTLs induced by administering a immunogenic composition for inducing an immune response to non-human animals and the like as an active ingredient, as well as the above-mentioned immunogenic composition for inducing an immune response. Preferable forms for these pharmaceutical agents or pharmaceutical composition are the forms which can be administered orally, intravenously, intraperitoneally, intranasally, intracutaneously, subcutaneously, intramascularly and the like. Effective doses to be administered can be determined accordingly by considering the types and compositions of such pharmaceutical agents or pharmaceutical compositions, the administration methods, the age and body weight of a patient, etc. It is preferable to administer them one or a few times a day. When administered orally, it is ordinarily administered through the formulations prepared by mixing with a carrier for formulation. Here, a substance which is usually used in the formulation field, and which does not react with the peptide of the present invention is used as a carrier for formulation.

Further, dosage forms can be particularly exemplified by tablets, capsules, granules, powder, syrup, suspension agent, suppository, ointment, cream, gel, patch, inhalant, injectable solution, etc. These formulations are prepared by ordinary protocols, and particularly, liquid formulation can be dissolved or suspended into water or other appropriate media when used. Tablets and granules may be coated by a known method. Injectable solution is prepared by dissolving the peptide of the present invention into water, however, it may also be dissolved into physiological saline or glucose solution according to need, and buffer agent or preservation agent may be further added. These formulations may also contain other ingredients of therapeutic value.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase entry of the pharmaceutical composition or agent into a preferred tissue site (such as for example a lipid delivery system).

The CTL recognition antigen or the antigen epitope thereof which can induce CTLs having an anti-tumor effect against HTLV-I tumors of the present invention can also be ingested as a functional food by compounding them into the following as food materials for infection control of HTLV-I and/or symptom improvement of HTLV-I-associated disease: baked goods either frozen or fresh such as puddings, chocolates, cookies, breads, cakes, jellies, crackers, and including pizza, biscuits, pies etc.; confectioneries such as sweet jellied azuki-bean pastes, etc.; breads and confectioneries such as frozen desserts, chewing gums, etc; noodles such as wheat noodles, buckwheat noodles, etc.; fish paste products such as steamed fish pastes, hams, fish meat sausages; various beverages such as yogurts, yogurt drinks, juice, cow milk, soy milk, alcoholic beverages, coffee, tea, natural leaf tea, oolong tea, isotonic drinks, etc.; condiments such as soybean pastes, soy sauce, dressing, mayonnaise, edulcorant, etc.; bean-curds; devil's tongue; various prepared food such as fish boiled in soy sauce, dumplings, croquettes, salads, nuts; dairy products including cheese, whipped desserts, and ice cream; meat products including sausages, fish, ham, pork and beef, such as joints of pork or beef; fresh and dried fruit; and snacks.

Similarly, the antigen or antigen epitopes can be added to compositions that can be combined with animal feed stock and/or water provisions, dog food, cat food, bird food, or rodent food. One of skill in the art will understand this method of administration is sometimes referred to as “bait dropping,” in which the pharmaceutical composition is included within the food and/or water of the organism to be vaccinated.

### INDUSTRIAL APPLICABILITY

CTLs induced by using protein, peptide, nucleic acid and the like of a CTL recognition antigen or an antigen epitope thereof which can induce CTLs having an anti-tumor effect against HTLV-I tumors as an immunogen, and by using an adjuvant obtained from the screening method of the present invention can strongly suppress the proliferation of HTLV-I-infected tumor cells in vivo. Therefore, protein, peptide, nucleic acid and the like of such CTL recognition antigen or such antigen epitope thereof which can induce CTLs having an anti-tumor effect against HTLV-I tumors are useful as a immunogenic composition for HTLV-I-associated diseases such as an adult T cell leukemia (ATL), etc., and CTLs induced by said protein, peptide, nucleic acid and the like are useful as a preventive and therapeutic agent for the above-mentioned HTLV-I-associated diseases.

The present invention will be further particularly explained hereinafter with reference to the examples, but the scope of the invention will not be limited to these examples.

### EXAMPLES

### Example 1

### Cell Lines

FPM1-V1AX (J. Virol. 73, 6031-6040, 1999) was established by proliferating immortalized rat T cell lines FPM1 established through the infection of HTLV-I with T cells of immunocompetent rats F344/N Jcl-rnu/+ (nu/+) (4-week-old female rats; Clea Japan, Inc.), in the body of a nude rat F344/N Jcl-rnu/rnu (nu/nu) (J. Virol. 73, 6436-6443, 1999). FPM-SV was established by transforming HTLV-I negative SV40 to nu/+rat-derived renal cell lines (J. Virol. 73, 6031-6040, 1999). An HTLV-I-infected T cell line established from splenocytes of WKAH rats (TARS-1; J. Exp. Med. 159, 1105-1116, 1984) was kindly provided by Dr. T. Yoshiki of Hokkaido University and used in the experiment. RT1.A¹/TARS-1 was established by transfecting RT1.A¹-expressing plasmid pRep10 into TARS-1 cells followed by in vitro selection with 400 μg/ml of hygromycin. Besides, pRep10 (Immunogenetics 39, 447, 1994) was kindly provided by Dr. S. Salgar (Miami Univ., FL) and used herein. The expression of RT1.A¹ in the above-mentioned RT1.A¹/TARS-1 cells was confirmed by immunofluorescence analysis. G14-Tax cell lines which are the cells that stably express G14 and Tax genes, i.e. the IL-2-dependent HTLV-I negative CD8⁺ T cell lines established from nu/+rats were prepared by the method as described previously (J. Virol. 74, 9610-9616, 2000). All the cell lines used herein were maintained in RPMI1640 containing 10% heat-inactivated FCS (Whittaker, Walkersville, Md.), penicillin, and streptomycin. 10 U/ml of recombinant human IL-2 (rhIL-2) (Shionogi Pharmaceutical & Co., Ltd.) was added to the culture medium RPMI1640 for maintaining G14 and G14-Tax.

### Example 2

### Preparation of Immune T cell Subset

4-week-old nu/+ rats were immunized with intraperitoneal administration of 2×10⁷ of FPM1-V1AX cells two times with a two-week interval. One week after the last immunization, splenic T cells were isolated and purified through a nylon wool column, then lymphocytes subsets were further purified using complement lysis method. To explain briefly, supernatants of splenic T cells were mixed with anti-rat CD8 monoclonal antibodies (R1-10B5) or anti-rat CD4 monoclonal antibodies (RTH-7), and incubated on ice for 30 minutes. After washed with 1% FCS-PBS, the above-mentioned cells were incubated in the culture medium RPMI1640 containing 2% rabbit serum (Cedarlane Laboratories Limited, Ontario, Canada) at 37° C. for 45 minutes, then washed two times with 10% FCS-RPMI 1640. Subsequently, it was confirmed by a flow cytometry analysis whether CD8⁺ or CD4⁺ T cells were condensed to on and above 98%.

### Example 3

### Anti-Tumor Effect of CD4+ or CD8⁺ T Cells

The present inventors previously reported that adoptive transfer of splenic T cells from rats immunized with HTLV-I-infected cells effectively inhibited the proliferation of malignant lymphomas in F344/N Jcl-rnu/rnu (nu/nu) rats wherein an HTLV-I-infected tumor cell line FPM1-V1AX was administered (J. Virol. 73, 6031-6040, 1999). Therefore, in order to clarify which subsets of immune T cells are directly required for tumor regression in vivo, anti-tumor effect against each subset of F344/N Jcl-rnu/rnu (nu/nu) rats was examined by using CD4+ or CD8⁺ cell groups isolated by the method described in Example 2. 2×10⁷ of FPM1-V1AX cell lines were subcutaneously inoculated into 4-week-old nu/nu female rats (Clea Japan, Inc.) that were simultaneously administered intraperitoneally with 107 of CD4⁺ T cells (▴), CD8⁺ T cells (▪), or total T cells (●) isolated in Example 2. Then suppressing effect on tumor proliferation was examined by measuring the size of each subcutaneous tumor with a caliper every other day. In these measurements, the longest surface length (mm; a) and width (mm; b) were determined by the above-mentioned measurement, and the tumor volume (V; mm³) was calculated using the formula (V=ab²/2) as described previously (J. Virol. 73, 6031-6040, 1999). Besides, 4-week-old nu/nu rats subcutaneously inoculated with FPM1-V1AX cell lines alone served as controls (◯).

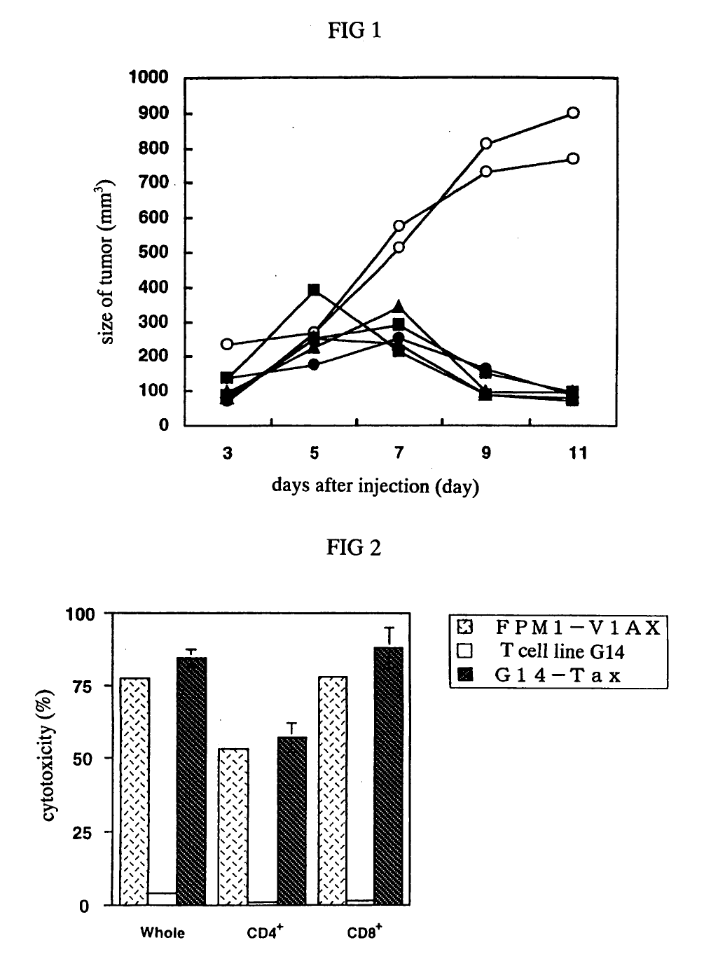

The results of the above are shown in FIG. 1. It was confirmed that both CD4+ and CD8⁺ T cells exhibited equivalent inhibitory effect on the growth of HTLV-I-infected tumor cell lines as well as total T cells. No metastatic tubercles were observed in the rats which underwent transfer of any subsets of the immune T cells, in contrast to the visible tubercles in the lungs and mediastinal lymph nodes of tumor-bearing rats.

In order to conduct further analysis, splenic T cells (CD4⁺ T cells, CD8⁺ T cells, or total T cells) were isolated respectively from each nu/nu rat on day 28 when complete tumor regression occurred by transferring immune T cells (CD4⁺ T cells, CD8⁺ T cells, or total T cells), then their cytotoxic activities against the respective target cells were examined. The above-mentioned various splenic T cells (5×10⁶ cells/well) were cocultured with formalin-fixed FPM1-V1AX cells (2×10⁶ cells/well) in 2 ml of 10% FCS-RPMI 1640 per well of a 24-well plate for 6 days. Cytotoxic activities against target cells were measured by ⁵¹Cr release assay for 6 hours as described previously (Immunology 14, 181-196, 1968) at the ratio of said cells (E; effector cells) and target cells (T; FPM1-V1AX, syngeneic T cell lines G14, or G14-Tax) (E/T) as 10, and calculated by the formula as described below. In addition, values (mean±SD) were evaluated from three independent experiments.

$$\frac{\begin{matrix}[ experimental {}^{51} Cr release - \\ spontaneous {}^{51} Cr release ]\end{matrix}}{\begin{matrix}[ maximum {}^{51} Cr release - \\ spontaneous {}^{51} Cr release ]\end{matrix}} \times 100 \% \qquad \text{Formula 1}$$

The results of the above are shown in FIG. 2. From these results, various T cells showed high cytotoxic activities against FPM1-V1AX and G14-Tax, however, they did not show a cytotoxic activity against HTLV-I negative G14 cells. In addition, it was notably recognized by flow cytometry that the splenic T cells isolated from nu/nu rats wherein the above-mentioned various immune T cells (CD4+ or CD8⁺ immune T cells) were transferred, were positive for CD4 or CD8 respectively. These results indicate that an HTLV-I-specific cytotoxic activity of CD4+ or CD8⁺ T cells is strongly involved in direct elimination of HTLV-I-infected tumor cells in vivo.

### Example 4

### Recombinant Vaccinia Virus

Subsequently, in order to examine viral antigens recognized by HTLV-I-specific CTLs, the following recombinant vaccinia viruses containing 4 types of HTLV-I genes provided by Dr. H. Shida, Hokkaido University (rvv; Embo J. 6, 3379-3384, 1987; J. Virol. 62, 3718-3728, 1988; Cell 55, 197-209, 1988) were used herein: rvv containing HTLV-I env gene (WR-env); rvv containing HTLV-I gag gene (WR-gag); rvv containing HTLV-I pX gene (WR-40X and WR-27X). WR-27X is a recombinant vaccinia virus that expresses p21X, p27rex, and p40tax, whereas WR-40X is the one that expresses p21X and p40tax. Each of the above-mentioned HTLV-I-rvv (WR-40X, WR-27X, WR-env, WR-gag) was infected with FPM-SV cells at an m.o.i. (multiplicity of infection) of 10 at 37° C. for 1 hour. After incubation, the cells were washed once with 10% FCS-RPMI 1640, then cultured in 10% FCS-RPMI 1640 at 37° C. for 12 hours. Said infected cells were labeled with a radioactive isotope in order to be used for the following example.

### Example 5

### HTLV-I Antigen Recognized by Rat CTLs

Splenic T cells isolated from nu/+ rats that had been intraperitoneally administered with FPM1-V1AX were stimulated with formalin-fixed FPM1-V1AX for 6 days, and then used as effector cells in the following example. As the target cells, FRM1-V1AX, FPM-SV, and various HTLV-I-rvv-infected FPM-SV cells (WR-40X, WR-27, WR-env, WR-gag) prepared in Example 4 were used. In addition, the cells wherein rvv which does not contain an HTLV-I gene are infected with FPM-SV cells (WR-HA) were used as controls. As a result of immunofluorescence analysis conducted for the above-mentioned HTLV-I-rvv-infected FRM-SV cells, these cells showed positive for MHC class I, but negative for MHC class II antigen. Therefore, these cells are considered to selectively express an antigen confined to MHC class I.

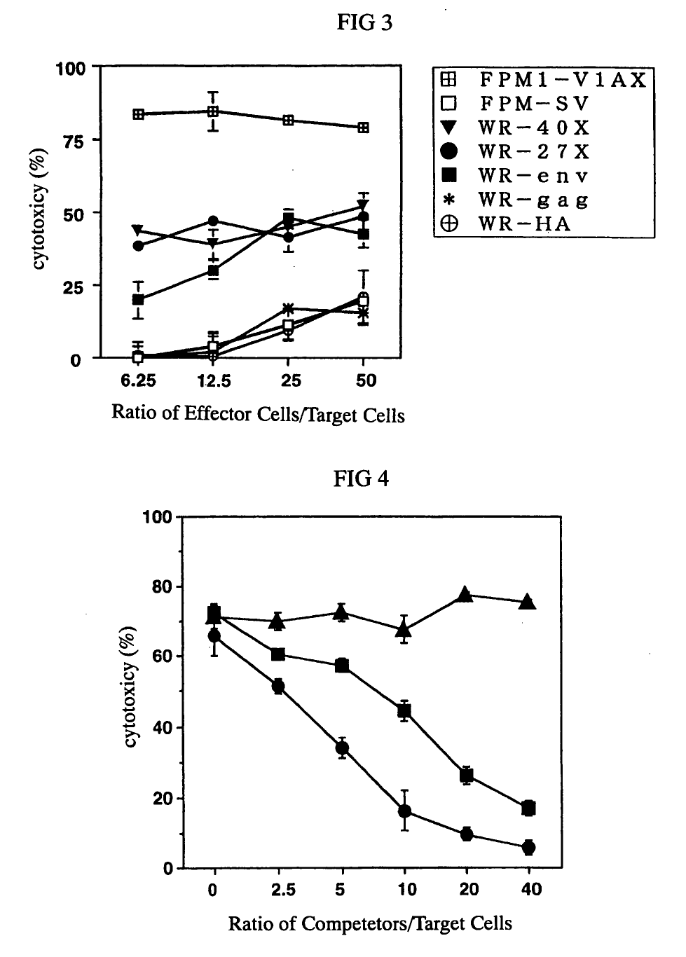

In the same way as described in Example 3, ⁵¹Cr release assay was conducted using the above-mentioned effector cells (E) and target cells (T) at each E/T ratio as shown in FIG. 3. Measurements (mean±SD) were evaluated from three independent experiments. The results are shown in FIG. 3. These results show that effector cells have high cytotoxicity against FPM1-V1AX, but not FPM-SV cells. Among HTLV-I-rvv-infected FPM-SV cells, WR40X- and WR-27X-infected cells, which commonly express HTLV-I tax, showed the highest sensitivity for effector cells. The target cells that express HTLV-I env (envelope) also showed sensitivity, but to a lesser degree. However, cytotoxic activities against FPM-SV cells that express HTLV-I gag were as low as the cytotoxic activities against target cells (WR-HA) as controls.

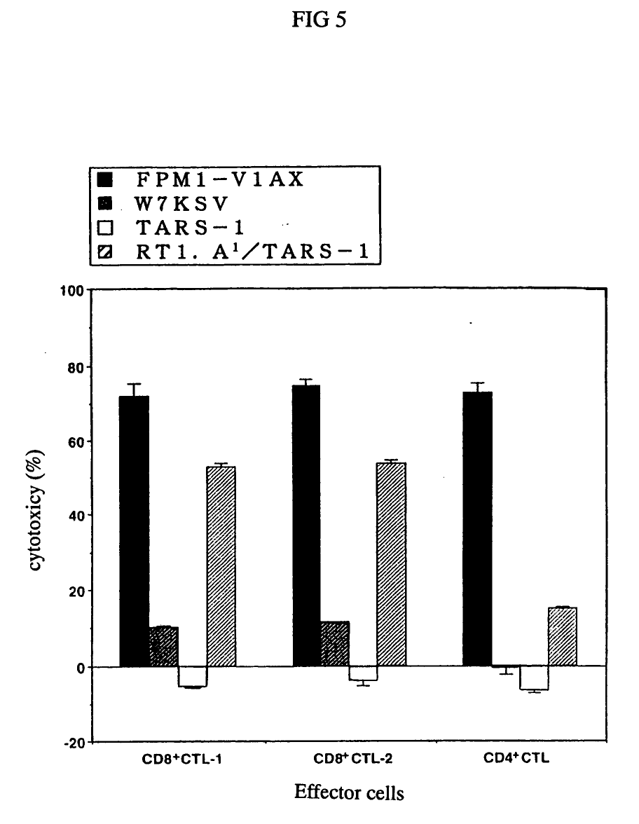

Subsequently, the inhibition of cytotoxicity of HTLV-I-specific CTL cell lines against target cells by unlabeled cells was examined. The CD8⁺ CTL cell lines which are more specific to HTLV-I Tax obtained by the long-term culture and the target cells ([³H]-TdR-labeled FPM1-V1AX:5×10³ cells/well) were plated on 96-well U-bottom plates at an E/T ratio of 10, then a competitor [unlabeled FPM1-V1AX (●), G14-Tax (▪), or G14 (▴)] was added at a competitor-to-target ratio as described in FIG. 4, and incubated at 37° C. for 6 hours. Thereafter, cells were harvested with a Micro 96 Harvester (Skatron), then the amount of remaining target cells was measured by a microplate β-counter (Micro Beta Plus), and the specific cytotoxicity (%) was calculated by the following formula. Values (mean±SD) were evaluated from three independent experiments. The cells were incubated with 3.7 MBq of [³H]-TdR per 10⁶ cells at 37° C. for 12 hours, then washed three times in advance were used as the above-mentioned target cells.

$$\frac{\begin{matrix}[ cpm without the presence of an effector - \\ cpm in the presence of an effector ]\end{matrix}}{[ cpm without the presence of an effector ]} \times 100 \% \qquad \text{Formula 2}$$

The results of the above are shown in FIG. 4. These results show that cytotoxic activities of CTL cell lines against FPM1-V1AX are notably inhibited by increasing unlabeled G14-Tax but not G14 cells. These results indicate that HTLV-I Tax is one of the major antigen specifically recognized by CTLs derived from nu/+ rats administered with syngeneic HTLV-I-infected cells in vivo.

### Example 6

### MHC Class I Restrain of HTLV-I-Specific Cytotoxic Activity in nu/+ Rats

For induction of HTLV-I-specific CTL cell lines (two types of CD8⁺ CTL cell lines and CD4⁺ CTL cell lines) in long-term culture, 2.5×10⁶ cells/well of splenic T cells were cocultured with the same number of formalin-fixed FPM1-V1AX cells in 10% FCS-RPMI 1640 wherein 20 U/ml of rhIL-2 was added, with periodical stimulation using formalin-fixed FPM1 cells every two weeks, and two types of CD8+CTL cell lines (CD8⁺ CTL-1 and CD8⁺ CTL-2) and CD4⁺ CTL cell lines were established. Then, the MHC class I restrain against cytotoxic activities was examined in the same way as Example 5 using such induced HTLV-I-specific CTL cell lines. As target cells, 4 cell lines labeled with [³H]-TdR in the same way as in Example 5 were used: nu/+rat-derived FPM1-V1AX that expresses a rat MHC class I molecule, RT1.A¹ X; WKAH rat-derived W7KSV that does not express RT1.A¹; WKAH rat-derived TARS-1 that does not express RT1.A¹; a cell line RT1.A¹/TARS-1 established by stably transfecting RT1.A¹ cDNA to TARS-1 cells.

The results of the above are shown in FIG. 5. Two types of CD8⁺ CTL cell lines (CD8⁺ CTL-1 and CD8⁺ CTL-2) notably dissolved RT1.A¹/TARS-1 cell but not TARS-1 cells, however, dissolution was not recognized in CD4⁺ CTL cell lines derived from nu/+ rats immunized with FPM1-V1AX cells. These findings show that the cytotoxic activity of CD8⁺ CTL cell lines specific to nu/+rat-derived HTLV-I Tax is restrained by RT1.A¹ of rat MHC class I.

### Example 7

### Identification of Recognition Epitope of HTLV-I-Specific CTLs

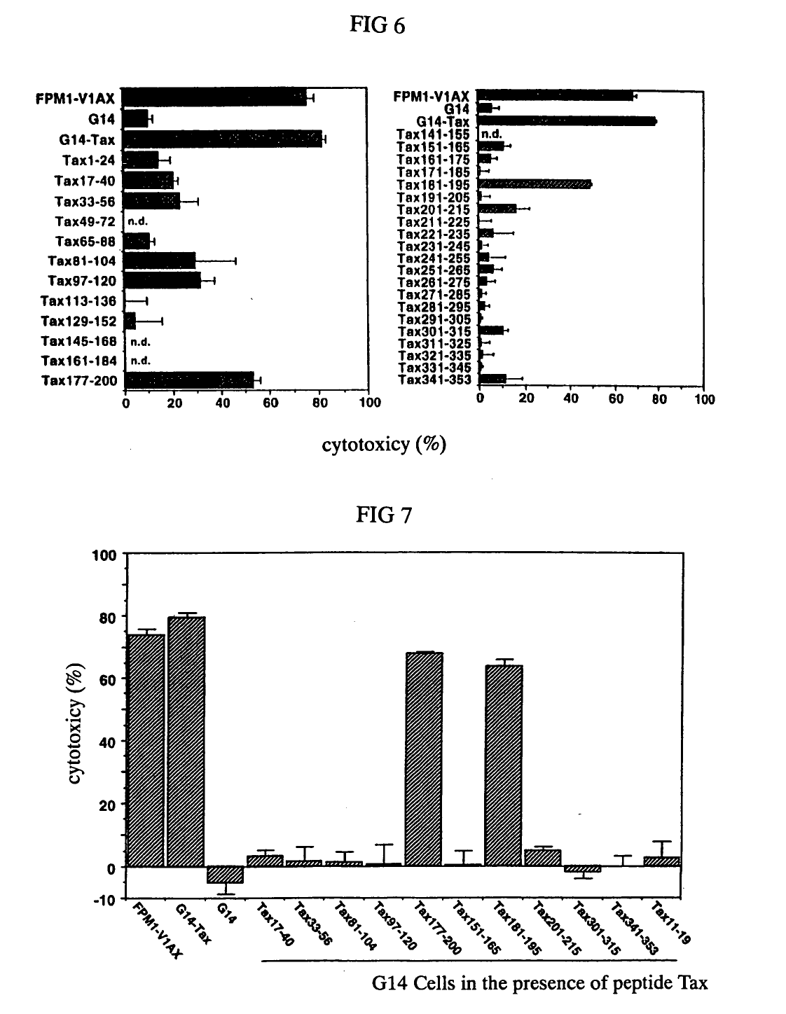

Peptide mapping analysis was conducted to identify target epitopes recognized by HTLV-I Tax-specific CD8⁺ CTLs obtained from nu/+ rats immunized with FPM1-V1AX. HTLV-I-specific CTL cell lines restrained by rat MHC class I (RT1.A¹) were established by repeating stimulation with formalin-fixed FPM1-V1AX every two weeks to splenic T cells derived from syngeneic immunocompetent rats immunized with nu/+ rat-derived HTLV-I-infected cell lines FPM1-V1AX. Some of a series of synthetic peptides corresponding to the amino acid sequences of HTLV-I Tax shown in FIGS. 6, 7 and 8 were synthesized using a solid phase peptide synthesis method on an automated peptide synthesizer (model PSSM-8; Shimazu Corporation, Kyoto, Japan) with chemicals and program cycles provided by the manufacturer, then separated from plastic using hydrogen fluoride, and purified to on and above 95% purity by a reverse-phase chromatography on an HPLC system (Model SPRINT; PE Biosystems Japan, Tokyo, Japan). In addition, 9 mer oligopeptides (Tax 179-187, 180-188, 181-189, 182-190, 183-191, 184-192, 185-193, 186-194, 187-195, and influenza Matrix 58-66) were synthesized by Hokudo Co. (Hokkaido, Japan) on commission and used herein.

For sensitization of the target cells to be used for the cytotoxic activity analysis, 29 partially overlapping synthetic peptides (15-24 mers; partial peptide in the Tax proteins shown in FIGS. 6 and 7) were added to target cells derived from syngeneic rats labeled with a radioactive isotope [³H]-TdR (G14 cells; 5×10³ cells/well) to a concentration of 10 μM each, and cultured at 37° C. for 1 hour, then the sensitivity for HTLV-I-specific CTL cell lines was measured by [³H]-TdR release assay for 6 hours. Values (mean±SD) were evaluated from three independent experiments. In addition, E/T was set at 10. The results are shown in FIGS. 6 and 7. These results show that the HTLV-I-specific CTL cell lines effectively dissolve target cells sensitized with peptide Tax 177-200 or Tax 181-195, and show low cytotoxic activity against target cells sensitized with peptide Tax 17-40, Tax 33-56, Tax 81-104, or Tax 97-120 (FIG. 6). It was confirmed that long-term-cultured CTL cell lines also notably dissolved target cells sensitized with Tax 177-200 or Tax 181-195 (FIG. 7). Significant cytotoxicity was not recognized in target cells sensitized with synthetic peptides other than Tax 177-200 or Tax 181-195.

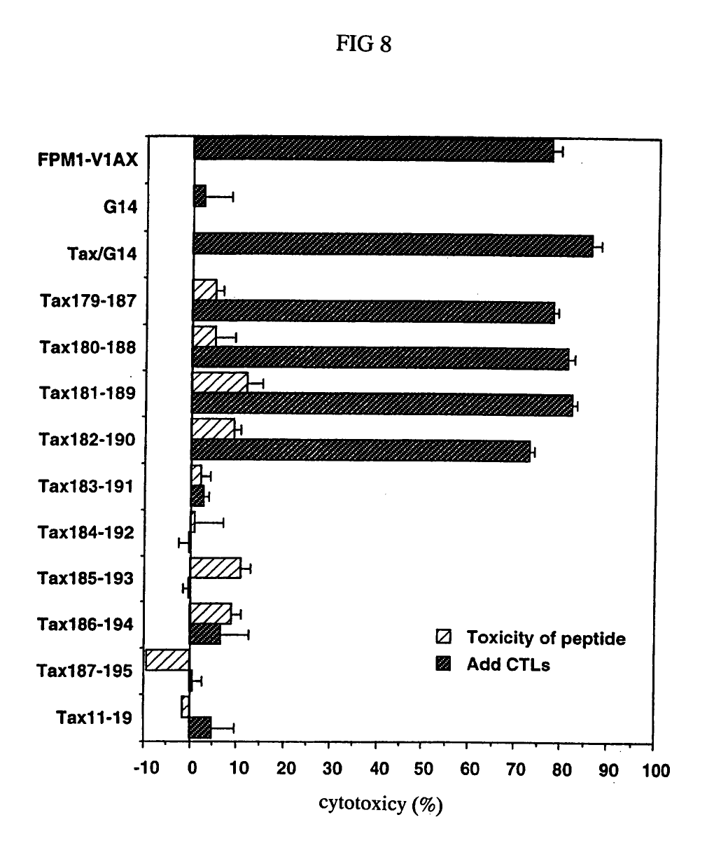

Based on the above-mentioned results, 10 different types of 9 amino acid synthetic peptides (9m peptide) included in the region of Tax 177-200 were synthesized, and detailed mapping of HTLV-I Tax-specific CTL epitope was conducted in the same method as mentioned above. The amino acid sequences of synthetic peptides used herein are shown in Table 1, and the results of cytotoxic activity analysis using these 9 amino acid synthetic peptides are shown in FIG. 8. These results show that the target cells sensitized with peptide Tax 180-188, Tax 181-189 show particularly strong CTLs sensitivity, then the target cells sensitized with Tax 179-187, Tax 182-190 exhibited CTL sensitivity for CTLs, however, the effect of Tax 179-187 varies among multiple experiments. The above-mentioned results indicated the possibility of the presence of the dominant epitope recognized by HTLV-I-specific CTLs in Tax 180-188 to Tax 182-190.

TABLE 1

| | |
|---|---|
| Peptide | Amino acid sequence |

| | |
|---|---|
| Tax 177-200 | GQLGAFLTNVPYKRIEELLYKISL |
| Tax 181-195 | AFLTNVPYKRIEELL |
| Tax 179-187 | LGAFLTNVP |
| Tax 180-188 | GAFLTNVPY |
| Tax 181-189 | AFLTNVPYK |
| Tax 182-190 | FLTNVPYKR |
| Tax 183-191 | LTNVPYKRI |
| Tax 184-192 | TNVPYKRIE |
| Tax 185-193 | NVPYKRIEE |
| Tax 186-194 | VPYKRIEEL |
| Tax 187-195 | PYKRIEELL |
| Tax 190-198 | RIEELLYKI |
| Tax 11-19 | LLFGYPVYV |
| (control peptide) | |

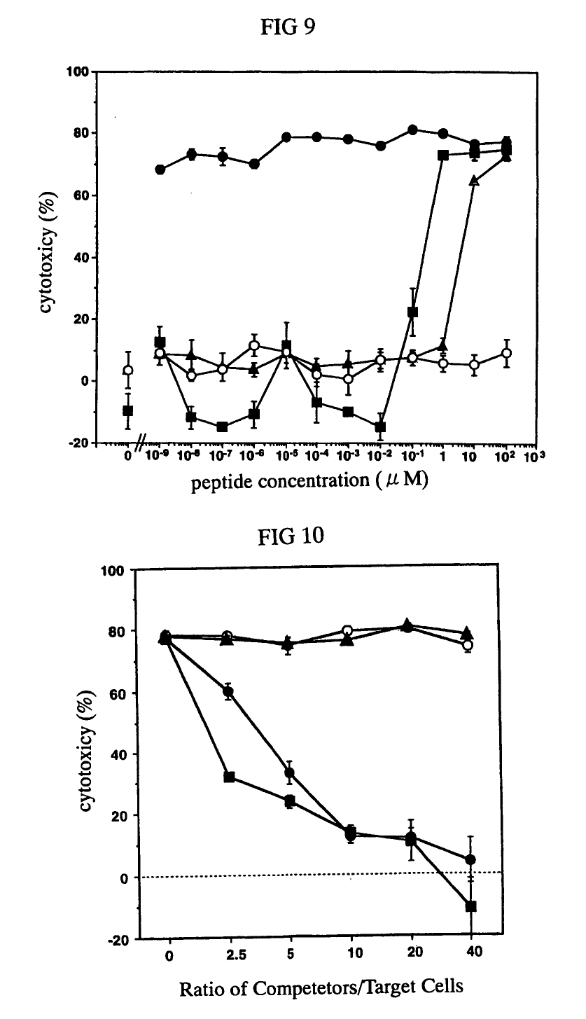

Subsequently, the peptide concentrations of Tax 180-188, Tax 181-189, Tax 182-190 necessary for inducing cytotoxic activity of CTLs were examined in order to clarify the dominant recognition epitope. Target cells G14 treated respectively with serially diluted peptides for 1 hour in advance, were cocultured with HTLV-I-specific CTL cell lines, and the CTL sensitivities of target cells were examined. The E/T ratio was at 10, and values (mean±SD) were evaluated from three independent experiments. The results are shown in FIG. 9. These results show that strong CTL sensitivity was confirmed in target cells sensitized with Tax 180-188 (●), and they show sensitivity to CTL even in extremely low peptide concentration, i.e. 10−³ pM. On the other hand, it was revealed that the concentration of 1 μM for Tax 181-189 (▪), and 10 μM for Tax 182-190 (▴) were necessary to induce cytotoxic activity of the same extent as Tax 180-188. No CTL sensitivity was recognized at any concentration in Tax 11-19 (◯), which was used as a control therein. The above-mentioned results revealed that the dominant recognition epitope of HTLV-I-specific CTLs restrained by RT1.A¹ was a peptide comprising 9 amino acids, i.e. Tax 180-188 (GAFLTNVPY: Seq. ID No.2).

### Example 8

### Recognition of Tax 180-188 by Tax-Specific CTLs of HTLV-I

In order to examine whether the peptide Tax 180-188 is dominant among the target epitopes in HTLV-I Tax recognized by HTLV-I-specific CTL cell lines, the cytotoxic activities of HTLV-I Tax-specific CTL cell lines against [³H]-TdR-labeled G14-Tax cell were measured in the presence of G14 cells and unlabeled G14-Tax cells (●) which had been sensitized in advance with unlabeled competitors such as, untreated G14 cells (◯), 10 μM of Tax 180-188 (▪), or Tax 11-19 (▴). The E/T ratio was at 10, and values (mean±SD) were evaluated from three independent experiments. The results are shown in FIG. 10. As a result, it was revealed that when G14 cells treated with 10 μM of Tax 180-188 (▪) were used, the cytotoxic activities against radiolabeled G14-Tax were completely inhibited when the competitor/Target cells ratio was at 40. The competitive effect of G14 cells treated with Tax 180-188 was at the same extent as the competitive effect of unlabeled G14-Tax (●). Untreated G14 cells (◯) or G14 cells treated with 10 μM of various peptides [Tax 181-189, Tax 182-190, and Tax 11-19 (▴)], scarcely affected the cytotoxic activities of HTLV-I Tax-specific CTL cell lines. These results indicate that the epitope Tax 180-188 is the dominant epitope recognized by HTLV-I Tax-specific CTL cell lines.

### Example 9

### Suppression Effect on Proliferation of HTLV-I-infected Tumor Cells of Tax 180-188 Recognition CTL Cell Lines In Vivo

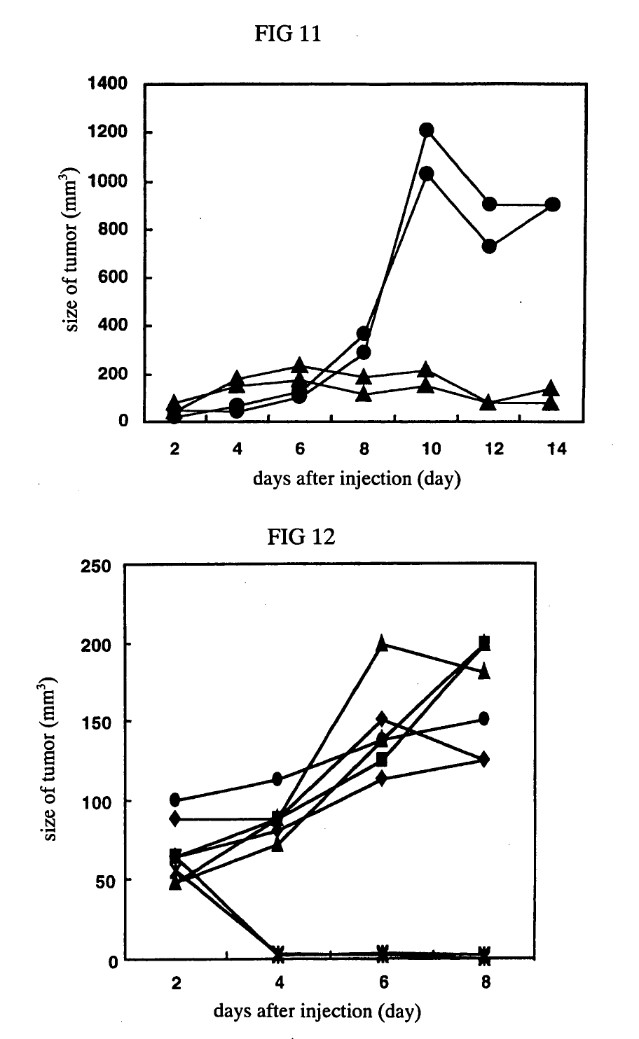

It was examined whether Tax 180-188 recognition CTL cell lines can possibly suppress the proliferation of HTLV-I-infected tumor cell FPM1-V1AX in rats in vivo. Suppression effect on tumor proliferation in rats wherein FPM1-V1AX was inoculated subcutaneously and CTL cell lines (107) that recognize Tax 180-188 were simultaneously administered intraperitoneally (▴) was observed by using nu/nu female rats subcutaneously inoculated with HTLV-I-infected tumor cell FPM1-V1AX (2×10⁷) alone as positive controls. Besides, the change of tumor proliferation was measured by measuring volume of each subcutaneous tumor by a caliper every other day as in Example 3, and the tumor volumes (V; mm³) were thus measured. Time courses of these subcutaneous tumor proliferations are shown in FIG. 11. The results show that subcutaneous tumors continued to proliferate in rats inoculated with FPM1-V1AX alone (●), whereas such proliferation was promptly rejected in rats simultaneously transfected with CTLS (▴). As a result of the above-mentioned, it was indicated that Tax 180-188 recognition CTL cell lines can possibly suppress in vivo proliferation of HTLV-I-infected tumor cells. In other words, there is a possibility that Tax 180-188 peptide is an important epitope as a tumor rejection antigen.

### Example 10

### Vaccine Effect of Tax 180-188 and ISS-ODN

Whether the antigen peptide determined as above-mentioned has actually become a tumor rejection antigen alone in vivo is extremely important in considering to what extent this antigen peptide can be applied as a tumor vaccine model. Subsequently, therefore, it was considered whether a peptide vaccine can induce Tax 180-188-specific CTLs in vivo, and whether such induced CTLs can show an anti-tumor effect in vivo. ISS-ODN (Immunostimulatory DNA sequences-oligodeoxynucleotide) containing CpG motif was used as an adjuvant, in order to induce Tax 180-188-specific CTLs more efficiently (Nat. Med. 3, 849-854, 1997). In addition, as an ISS-ODN [ESPEC OLIGO SERVICE; 5′-TGACTGTGAACGTTCGAGATGA-3′ (Seq. ID No.5)], the one synthesized as phosphorothioate single-stranded oligonucleotides were used. 100 μg of Tax 180-188 synthetic peptide (▪), 10 mmol of ISS-ODN (▴), 100 μg of Tax 180-188 mixed with 10 nmol of ISS-ODN (*), or 100 μg of Influenza A matrix (GILGFVFTL; Seq. ID No.6) peptide comprising the 58-66^{th} amino acid sequences mixed with 10 nmol of ISS-ODN (♦) were respectively mixed in 200 μl of physiological saline and administered subcutaneously into 4-week-old nu/+ female rats, and they were immunized again after two weeks on the same condition. Besides, rats subcutaneously administered with physiological saline alone were used as controls (●). Two weeks after the last immunization, splenic T cells (107) were separated from each rat, then administered intraperitoneally to 4-week-old nu/nu female rats, that were simultaneously administered intraperitoneally with HTLV-I-infected tumor cells, FPM1-V1AX (2×10⁷). The suppression effect on tumor proliferation at the inoculated sites were measured in the same manner as Example 3, i.e. by measuring the size of each subcutaneous tumor by a caliper every other day, thus the volumes of tumors were measured (V; mm³).

The results of the above are shown in FIG. 12. As a result, the suppression effect on tumor proliferation was strongly suppressed in a group wherein splenic T cells derived from rats immunized with Tax 180-188 and ISS-ODN (*) were transfected, however, such suppression effect on tumor proliferation was not recognized in the group transfected with splenic T cells derived from rats immunized respectively with the following: Tax 180-188 peptide only (▪); ISS-ODN only (▴); Influenza A matrix 58-66 and ISS-ODN (♦). As a result of the above-mentioned, it is revealed to be possible to induce Tax 180-188-specific CTLs to immunocompetent rats by using an appropriate adjuvant, and that such induced CTLs can strongly suppress the proliferation of HTLV-I-infected tumor cells in vivo. These results show that Tax 180-188 can act independently as a tumor rejection antigen, and that a vaccine wherein Tax 180-188 peptide and ISS-ODN are used together efficiently induce T cell immunity and protect from HTLV-I-infected tumors in vivo, and that the present experimental system is extremely useful as a developmental model of a vaccine.

The invention is further described by the following numbered paragraphs:

1. A screening method for a CTL recognition antigen or an antigen epitope thereof which can induce cytotoxic T lymphocytes (CTLs) having an anti-tumor effect against HTLV-I tumors, wherein CTLS induced by a test substance is administered to a non-human animal model of HTLV-I-associated disease, and the change of tumors in said non-human animal is measured and assessed.

2. The screening method for a CTL recognition antigen or an antigen epitope thereof which can induce CTLs having an anti-tumor effect against HTLV-I tumors according to paragraph 1, wherein the non-human animal model of HTLV-I-associated disease is a non-human animal model of adult T cell leukemia.

3. The screening method for a CTL recognition antigen or an antigen epitope thereof which can induce CTLs having an anti-tumor effect against HTLV-I tumors according to paragraph 1 or 2, wherein the non-human animal is a rat.

4. A screening method for a CTL recognition antigen or an antigen epitope thereof which can induce CTLs having an anti-tumor effect against HTLV-I tumors, wherein CTLs induced by a test substance is contacted with HTLV-I-infected tumor cell lines, and the cytotoxic activity of said CTLs is measured and assessed.

5. A screening method for a CTL recognition antigen or an antigen epitope thereof which can induce CTLs having an anti-tumor effect against HTLV-I tumors, wherein target cells sensitized with a test substance or target cells that express a test substance are contacted with HTLV-I-specific CTL cell lines, and the cytotoxic activity of said HTLV-I-specific CTL cell lines is measured and assessed.

6. The CTL recognition antigen or the antigen epitope thereof which can induce CTLs having an anti-tumor effect against HTLV-I tumors, which is obtained by the screening methods according to any of paragraphs 1 to 5.

7. The antigen epitope according to claim 6, wherein the antigen epitope is a peptide comprising an amino acid sequence shown by Seq. ID No. 2.

8. A screening method for an adjuvant that enhances an anti-tumor effect against HTLV-I tumors, wherein CTLs induced by using the CTL recognition antigen or the antigen epitope thereof according to paragraph 6 and a test adjuvant are administered to a non-human animal model of HTLV-I-associated diseases and the change of tumors in said non-human animals is measured and assessed.

9. The screening method for an adjuvant that enhances an anti-tumor effect against HTLV-I tumors according to paragraph 8, wherein the non-human animal model of HTLV-I-associated disease is a non-human animal model of adult T cell leukemia.

10. The screening method for an adjuvant that enhances an anti-tumor effect against HTLV-I tumors according to paragraph 8 or 9, wherein the non-human animal is a rat.

11. A screening method for an adjuvant that enhances an anti-tumor effect against HTLV-I tumors, wherein CTLs induced by using the CTL recognition antigen or the antigen epitope thereof according to paragraph 6 and a test adjuvant are contacted with HTLV-I-infected tumor cell lines, and the cytotoxic activity of said CTLs is measured and assessed.

12. The screening method for an adjuvant that enhances an anti-tumor effect against HTLV-I tumors according to any of paragraphs 8 to 11, wherein the antigen epitope peptide according to paragraph 7 is used as the CTL recognition antigen or the antigen epitope thereof according to paragraph 6.

13. A vaccine for inducing an immune response containing a CTL recognition antigen or an antigen epitope thereof which can induce CTLs having an anti-tumor effect against HTLV-I tumors and which can be obtained by the screening method according to any of paragraph 1 to 5 as an active ingredient.

14. A vaccine for inducing an immune response containing a DNA that encodes a CTL recognition antigen or an antigen epitope thereof which can induce CTLs having an anti-tumor effect against HTLV-I tumors obtained by the screening method according to any of paragraphs 1 to 5 as an active ingredient.

15. The vaccine for inducing an immune response according to paragraph 13 or 14, wherein the CTL recognition antigen is an HTLV-I Tax protein shown by Seq. ID No.1.

16. The vaccine for inducing an immune response according to paragraph 13 or 14, wherein the CTL recognition antigen is a protein comprising an amino acid sequence wherein one or a few amino acids are deleted, substituted, or added in the amino acid sequence shown by Seq. ID No.1, which can induce CTLs having an anti-tumor action against HTLV-I tumors.

17. The vaccine for inducing an immune response according to paragraph 13 or 14, wherein the CTL recognition antigen epitope is a peptide comprising the amino acid sequence shown by Seq. ID No.2.

18. The vaccine for inducing an immune response according to paragraph 13 or 14, wherein the CTL recognition antigen epitope is a peptide comprising an amino acid sequence wherein one or a few amino acids are deleted, substituted, or added in the amino acid sequence shown by Seq. ID No. 2, which can induce the CTLs having an anti-tumor action against HTLV-I tumors.

19. The vaccine for inducing an immune response according to any of paragraphs 13 to 18, further containing an adjuvant that enhances an anti-tumor effect against HTLV-I tumors.

20. The vaccine for inducing an immune response according to paragraph 19, wherein the adjuvant which can be obtained by the screening method according to any of paragraphs 8 to 12 is an adjuvant that enhances an anti-tumor effect against HTLV-I tumors.

21. The vaccine for inducing an immune response according to paragraph 20, wherein the adjuvant that enhances an anti-tumor effect against HTLV-I tumors which can be obtained by the screening method according to any of paragraphs 8 to 12 is ISS-ODN.

22. An HTLV-I recognition CTL induced by the vaccine for inducing an immune response according to any of paragraphs 13 to 21.

23. A pharmaceutical composition containing the HTLV-I recognition CTL according to paragraph 22 as an active ingredient.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

We claim:

1. A method of screening for a cytotoxic T-lymphocyte (CTL) recognition antigen or an antigen epitope thereof that can induce CTLs having an anti-tumor effect against HTLV-I tumors, wherein:
(a) CTLs induced by a test substance are administered to a non-human animal model of HTLV-I associated disease, and the change of tumors in said non-human animal is measured and assessed; or
(b) CTLs induced by a test substance are contacted with HTLV-I infected tumor cell lines and the cytotoxic activity of said CTLs is measured and assessed; or
(c) target cells sensitized with a test substance or target cells that express a test substance are contacted with HTLV-I specific CTL cell lines, and the cytotoxic activity of said HTLV-I specific CTL cell lines is measured and assessed.

2. The method of claim 1, wherein the HTLV-I associated disease is adult T-cell leukemia.

3. The method of claim 1, wherein the non-human animal is a rat.

4. A CTL recognition antigen or an antigen epitope thereof that can induce CTLs having an anti-tumor effect against HTLV-I tumors, which is obtained by the screening method of claim 1.

5. The antigen epitope of claim 4, wherein the antigen epitope is a peptide comprising an amino acid sequence of SEQ ID NO:2.

6. A method of screening for an adjuvant that enhances an anti-tumor effect against HTLV-I tumors, wherein:
(a) CTLs induced by using the CTL recognition antigen or antigen epitope of claim 4 and a test adjuvant are administered to a non-human animal model of HTLV-I associated disease and the change or tumors in said non-human animals is measured and assessed; or
(b) CTLs induced by using the CTL recognition antigen or antigen epitope of claim 4 and a test adjuvant are contacted with HTLV-I infected tumor cell lines, and the cytotoxic activity of said CTLs is measured and assessed.

7. The method of claim 6, wherein the HTLV-I associated disease is adult T-cell leukemia.

8. The method of claim 6, wherein the non-human animal is a rat.

9. An immunogenic composition for inducing an immune response containing a CTL recognition antigen or an antigen epitope thereof which can induce CTLs having an anti-tumor effect against HTLV-I tumors and which can be obtained by the method of claim 1.

10. An immunogenic composition for inducing an immune response containing a nucleic acid that encodes a CTL recognition antigen or an antigen epitope thereof which can induce CTLs having an anti-tumor effect against HTLV-I tumors obtained by the method of claim 1.

11. The immunogenic composition of claim 9, wherein the CTL recognition antigen comprises the amino acid sequence of SEQ ID NO:1.

12. The immunogenic composition of claim 10, wherein the CTL recognition antigen comprises the amino acid sequence of SEQ ID NO:1.

13. The immunogenic composition of claim 11, wherein at least one amino acid is deleted, substituted, or added.

14. The immunogenic composition of claim 12, wherein at least one amino acid is deleted, substituted, or added.

15. The immunogenic composition of claim 9, wherein the CTL recognition antigen is a peptide comprising the amino acid sequence of SEQ ID NO:2.

16 The immunogenic composition of claim 10, wherein the CTL recognition antigen is a peptide comprising the amino acid sequence of SEQ ID NO:2.

17. The immunogenic composition of claim 15, wherein at least one amino acid is deleted, substituted, or added.

18. The immunogenic composition of claim 16, wherein at least one amino acid is deleted, substituted, or added.

19. The immunogenic composition of claim 9, further comprising an adjuvant that enhances an anti-tumor effect against HTLV-I.

20. The immunogenic composition of claim 10, further comprising an adjuvant that enhances an anti-tumor effect against HTLV-I.

21. The immunogenic composition for inducing an immune response containing a CTL recognition antigen or an antigen epitope thereof which can induce CTLs having an anti-tumor effect against HTLV-I tumors and which can be obtained by the method of claim 1, further comprising an adjuvant that enhances an anti-tumor effect against HTLV-I, wherein the adjuvant is obtained by the method of screening for an adjuvant that enhances an anti-tumor effect against HTLV-I tumors, wherein:
(a) CTLs induced by using the CTL recognition antigen or antigen epitope that can induce CTLs having an anti-tumor effect against HTLV-I tumors, which is obtained by the screening method of claim 1 and a test adjuvant are administered to a non-human animal model of HTLV-I associated disease and the change or tumors in said non-human animals is measured and assessed; or
(b) CTLs induced by using the CTL recognition antigen or antigen epitope that can induce CTLs having an anti-tumor effect against HTLV-I tumors, which is obtained by the screening method of claim 1 and a test adjuvant are contacted with HTLV-I infected tumor cell lines, and the cytotoxic activity of said CTLs is measured and assessed.

22. The immunogenic composition for inducing an immune response containing a nucleic acid that encodes a CTL recognition antigen or an antigen epitope thereof which can induce CTLs having an anti-tumor effect against HTLV-I tumors obtained by the method of claim 1, further comprising an adjuvant that enhances an anti-tumor effect against HTLV-I, wherein the adjuvant is obtained by the method of screening for an adjuvant that enhances an anti-tumor effect against HTLV-I tumors, wherein:
(a) CTLs induced by using the CTL recognition antigen or antigen epitope that can induce CTLs having an anti-tumor effect against HTLV-I tumors, which is obtained by the screening method of claim 1 and a test adjuvant are administered to a non-human animal model of HTLV-I associated disease and the change or tumors in said non-human animals is measured and assessed; or
(b) CTLs induced by using the CTL recognition antigen or antigen epitope that can induce CTLs having an anti-tumor effect against HTLV-I tumors, which is obtained by the screening method of claim 1 and a test adjuvant are contacted with HTLV-I infected tumor cell lines, and the cytotoxic activity of said CTLs is measured and assessed.

23. The immunogenic composition of claim 19, wherein the adjuvant is ISS-ODN.

24. The immunogenic composition of claim 20, wherein the adjuvant is ISS-ODN

25. An HTLV-I recognition CTL induced by the immunogenic composition of claim 9.

26. An HTLV-I recognition CTL induced by the immunogenic composition of claim 10.

27. A pharmaceutical composition comprising the HTLV-I recognition CTL of claim 25.

28. A pharmaceutical composition comprising the HTLV-I recognition CTL of claim 25.


SEQUENCE LISTING (6 sequences)

SEQ ID NO: 1 - protein, 353 aa, Human T-cell lymphotropic virus type 1
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEELLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHSLHLLFEEYTNIPISLLFNEKEADDNDHEPQISPGGLEPPSEKHFRETEV

SEQ ID NO: 2 - protein, 9 aa, Human T-cell lymphotropic virus type 1
GAFLTNVPY

SEQ ID NO: 3 - protein, 9 aa, Human T-cell lymphotropic virus type 1
AFLTNVPYK

SEQ ID NO: 4 - protein, 9 aa, Human T-cell lymphotropic virus type 1
FLTNVPYKR

SEQ ID NO: 5 - DNA, 22 bp, Artificial Sequence
  Description of Artificial Sequence adjuvant ISS-ODN
tgactgtgaacgttcgagatga

SEQ ID NO: 6 - protein, 9 aa, Influenza A virus
GILGFVFTL